(19) United States
(12) Patent Application Publication
(10) Pub. No.: US 2016/0354251 A1
(43) Pub. Date: Dec. 8, 2016

(54) EARPLUG STRUCTURE

(71) Applicant: UNIQUE SAFETY EQUIPMENT CO., LTD., NEW TAIPEI CITY (TW)

(72) Inventor: LIN-GO CHEN, NEW TAIPEI CITY (TW)

(21) Appl. No.: 14/729,038

(22) Filed: Jun. 2, 2015

(51) Int. Cl. A61F 11/08

(52) U.S. Cl. CPC A61F 11/08

(57) ABSTRACT

An earplug structure being a columnar body has a sound insulation portion at an end and a holding portion at the other end. The sound insulation portion includes first to fourth flaps which are bowl-shaped structures, and the first flap has a surface curvature from −6.275 to 5.167, and the second flap has a surface curvature from −6.211 to −4.233, and the third flap has a surface curvature from −4.53 to −4.335, and the fourth flap has a surface curvature from −3.963 to −3.872, and the first, second, third and fourth flaps come with different sizes that increase gradually from the first flap to the fourth flap. The flaps are designed with a bowl-shaped curvature which increases the contact surface between the earplug structure and a user's auditory canal to achieve the effects of improving the close contact effectively and insulating the sound completely.

### BACKGROUND OF THE INVENTION

1. Field of the Invention

The present invention relates to the field of earplugs, and more particularly to an earplug structure with a high adaptation and capable of preventing the earplug structure from falling out from the user's ear effectively, and facilitating the user to find the earplug that falls out accidentally.

2. Description of the Related Art

At present, earplugs become an important necessary tool generally used by people working in a noisy environment, and the earplugs are plugged into a user's auditory canal and contacted tightly with the user's external auditory meatus for insulating and preventing sounds from entering into the user's middle ear and inner ear, so as to achieve the sound insulation effect and provide a quiet and peaceful rest or working environment to the user. With reference to FIG. 1 for a schematic view of a conventional earplug structure, the conventional earplug structure comprises a cylindrical earplug body 1, a plurality of earplug plates 11 formed on the earplug body 1, a hand holding portion 12 disposed at an end of the earplug body 1, and a hollow slotted hole 13 formed from the hand holding portion 12 to the end of the earplug body 1. Person working in a noisy environment usually wear such earplugs to protect their ears while working.

Due to the structure and shape, the conventional earplug is usually not in a close contact with the user's auditory canal, and thus causing a sound leakage easily, letting the earplug fall from the auditory canal easily, and just reducing the noise but not insulating the sound completely in a very noisy environment. In addition, the conventional earplug comes with a relatively small volume, so that users may not be able to find the earplug easily when it drops out from the user's ear accidentally. In view of the aforementioned problems, the inventor of the present invention based on years of experience to conduct extensive researches and experiments, and finally developed an earplug structure of the invention to overcome the problems of the prior art.

### SUMMARY OF THE INVENTION

Therefore, it is a primary objective of the present invention to provide an earplug structure capable of contacting with the user's auditory canal closely and preventing sound leakage effectively to achieve the effect of insulating sound completely.

A secondary objective of the present invention is to provide an earplug structure that allows the user to locate the position of the earplug structure quickly when it drops out from the user's ear cannel into a fluid such as a solution, food, or a space between a machine and the floor.

To achieve the aforementioned and other objectives, the present invention provides an earplug structure, which is a columnar body having a sound insulation portion disposed at an end of the earplug structure and a holding portion disposed at the other end of the earplug structure, characterized in that the sound insulation portion includes a first flap, a second flap, a third flap and a fourth flap which are bowl-shaped structures, and the first flap has a surface curvature from −6.275 to 5.167, and the second flap has a surface curvature from −6.211 to −4.233, and the third flap has a surface curvature from −4.53 to −4.335, and the fourth flap has a surface curvature from −3.963 to −3.872, and the first flap, the second flap, the third flap and the fourth flap come with different sizes that increase gradually from the first flap to the fourth flap, and the earplug structure has a connecting hole formed at an end of the holding portion, and the holding portion is doped with a metal powder to facilitate users to search for the earplug structure by using a metal detector.

Wherein, the first flap and the second flap, the second flap and the third flap, and the third flap and the fourth flap have a gap of 4 mm-4.5 mm apart, and the first flap and the second flap, the second flap and the third flap, and the third flap and the fourth flap have a volume ratio ranging from 1:1.25 to 1:1.43 to fit the shape of the user's auditory canal and make the wearing more comfortable to the user.

In addition, the present invention further comprises a sling, such that when a pair of earplug structures is used, both ends of the sling are inserted and fixed into the connecting hole of each of the earplug structures. The sling is provided for hanging the earplug structure to the user's neck to prevent the earplug structure from dropping to the floor or falling into a liquid when the earplug structure falls out from the user's ear.

The flaps are designed with a bowl-shaped curvature which increases the contact surface between the earplug structure and a user's auditory canal to achieve the effects of improving the close contact effectively and insulating the sound completely. In addition, the holding portion is doped with a metal powder, so that even if the earplug structure falls into a liquid and at a position which is difficult to be found visually, or food or a crevice, a metal detector can be used to locate the position where the earplug structure is dropped.

### BRIEF DESCRIPTION OF THE DRAWINGS

FIG. 1 is a schematic view of a conventional earplug structure;

FIG. 2 is a perspective view of a preferred embodiment of the present invention;

FIG. 3 is a planar view of a preferred embodiment of the present invention;

FIG. 4 is a schematic view of an application of a preferred embodiment of the present invention;

FIG. 5 is a schematic view of another application of a preferred embodiment of the present invention; and

FIG. 6 is a schematic view of searching for an earplug fallen out from a user's ear in accordance with a preferred embodiment of the present invention.

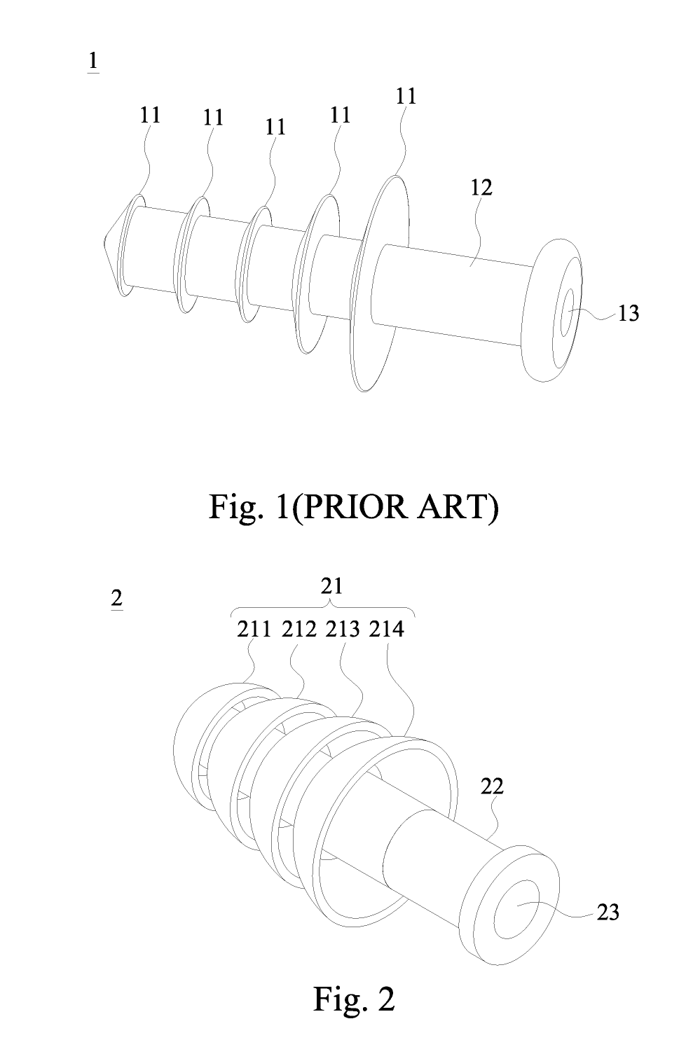

### DETAILED DESCRIPTION OF THE PREFERRED EMBODIMENTS

The technical content of the present invention will become apparent with the detailed description of preferred embodiments and the illustration of related drawings as follows. It is noteworthy that same numerals are used for representing the same respective elements respectively for simplicity.

With reference to FIG. 2 for a perspective view of an earplug structure in accordance with a preferred embodiment of the present invention, the earplug structure 2 is a columnar body having a sound insulation portion 21 disposed at an end of the earplug structure 2 and a holding portion 22 disposed at the other end of the earplug structure 2, characterized in that the sound insulation portion 21 includes a first flap 211, a second flap 212, a third flap 213 and a fourth flap 214, and the first flap 211, the second flap 212, the third flap 213 and the fourth flap 214 are made of a thermoplastic polyester plastic material and all are bowl-shaped structures, and the first flap 211 has a surface curvature from −6.275 to 5.167, and the second flap 212 has a surface curvature from −6.211 to −4.233, and the third flap 213 has a surface curvature from −4.53 to −4.335, and the fourth flap 214 has a surface curvature from −3.963 to −3.872, and the first flap 211, the second flap 212, the third flap 213 and the fourth flap 214 come with different sizes that increase gradually from the first flap 211 to the fourth flap 214, and the earplug structure 2 has a connecting hole 23 formed at an end of the holding portion 22. When a user wears the earplug structure, the user inserts the first flap 211 into the user's auditory canal and then inserts the second flap 212, the third flap 213 and the fourth flap 214 arranged from the inside to the outside.

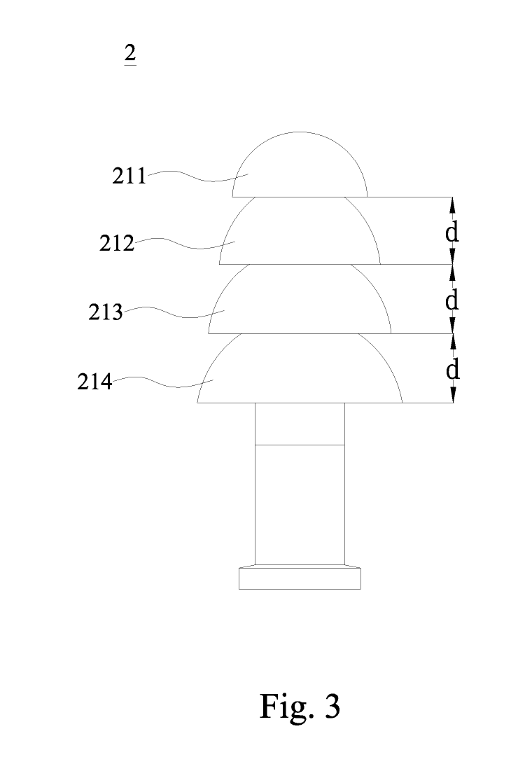

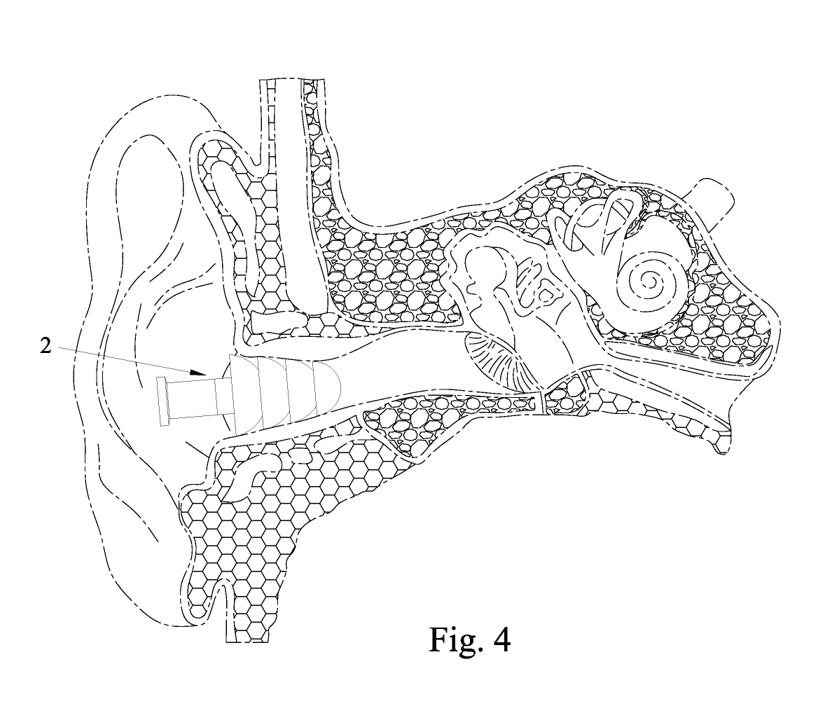

With reference to FIGS. 3 and 4 for a planar view of a preferred embodiment of the present invention and a schematic view of an application of a preferred embodiment of the present invention respectively, the first flap 211 and the second flap 212, the second flap 212 and the third flap 213, and the third flap 213 and the fourth flap 214 have a gap d falling within a range of 4 mm-4.5 mm; and a volume ratio of the first flap 211 to the second flap 212, the second flap 212 to the third flap 213, and the third flap 213 to the fourth flap 214 is falling within a range from 1:1.25 to 1:1.43. In actual practices, a better wearing comfort is achieved if the volume ratio falls within this range. For any ratio greater than the aforementioned range, the user may have an uncomfortable swollen feeling. For any ratio smaller than the aforementioned ranged, the close contact with the user's auditory canal may be insufficient which will result in a sound leakage.

When the user wants to use the earplug structure of the present invention, the user plugs the earplug body 2 into the user's auditory canal as shown in FIG. 4. Now, the volumes of the first flap 211 to the fourth flap 214 gradually increase and the flaps 211, 212, 213, 214 are made of plastic material, so that the flaps can fit different shapes of auditory canals, and the gap d between the flaps allows the volume of the flaps to increase significantly to fit the auditory canal with an externally increasing size. In addition, the first flap 211 to the fourth flap 214 are designed with a bowl-shaped curvature, so that when the earplug structure is plugged into the user's auditory canal, the contact surface between with the auditory canal is increased to improve the close contact of the present invention and improve the sound insulation effect. With the design of the surface curvatures of the first flap 211 to the fourth flap 214, the gap between the maximum curvature and the minimum curvature of the fourth flap 214 is the smallest which is similar to a spherical surface, so that after a sound wave is incident into the fourth flap 214, a vast majority of the sound wave is reflected to the outside, and a portion of the remaining sound wave will be absorbed by the soft plastic material, and the other portion of the remaining sound wave will be transmitted to the third flap 213 through vibration. Since there are four flaps in the present invention, therefore when the sound wave is transmitted from one flap to the other through vibration, the flaps can absorb some of the vibration energies. Actual tests show that most of the vibration energies will be absorbed when the sound wave (which is not too large) is transmitted from the fourth flap 214 to the first flap 211, and the remaining energy is insufficient to transmit the sound wave from the first flap 211 to the user's eardrum, and the close contact of the earplug structure of the present invention is capable of insulating and preventing sound waves from entering into the user's auditory canal or vibrating the user's eardrum.

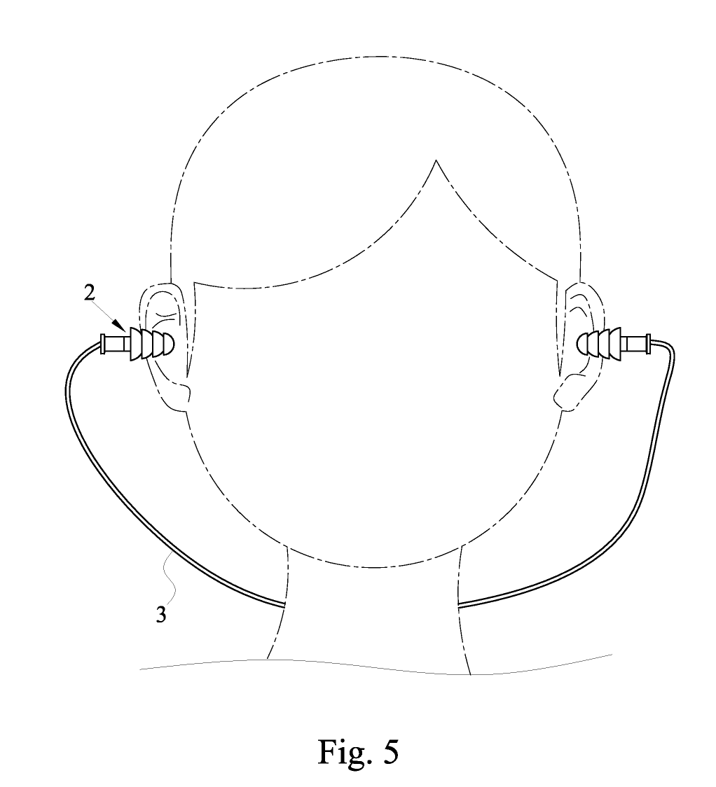

With reference to FIG. 5 for a schematic view of another application of a preferred embodiment of the present invention, a pair of earplug structures of the present invention are generally used, and the earplug structure of the present invention further comprises a sling 3 inserted and fixed into the connecting hole 23 of each of the earplug structures 2 and provided for hanging the earplug structures 2 to the user's neck, so that the earplug structures 2 will not fall onto the ground or into a liquid when the user shakes his/her body vigorously, and the sling 3 keeps the earplug structures 2 to be hung on the user's neck.

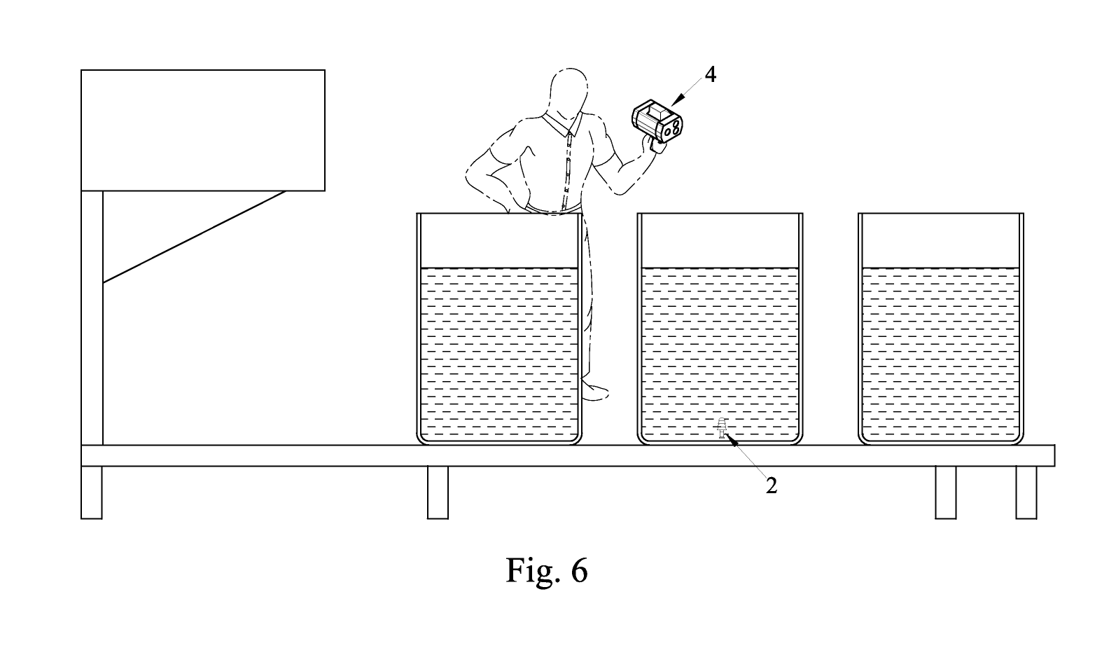

With reference to FIG. 6 for s a schematic view of searching for an earplug fallen out from a user's ear in accordance with a preferred embodiment of the present invention, the holding portion 22 of the earplug structure 2 of the present invention is doped with a metal powder, wherein the metal powder of this embodiment is iron powder. In FIG. 6, if the earplug structure 2 falls out from the user's auditory canal to the floor, food or a crevice, it will be difficult for the user to find the earplug structure 2 without the metal powder or it will take much time for the user to search for the earplug structure 2 particularly in a large space such as a factory. With the doped iron powder of the present invention, the user can locate the position of the earplug structure 2 by using a metal detector 4, so as to reduce the searching time and prevent a product or food containing the earplug structure 2 from being delivered and sold to consumers.

In addition, the surface of the earplug structure 2 of the present invention has three colored areas, and the first colored area covers the surface of the first flap 211 and the second flap 212, and the second colored area covers the surface of the third flap 213 and the fourth flap 214, and the third colored area covers the surface of the holding portion 22. These three colored areas are coated with different colors. For example, the first colored area is red, the second colored area is white, and the third colored area is blue. Therefore, the earplug structure 2 of the present invention becomes more prominent in a place with simple color such as the factory, and the fallen earplug structure 2 can be found more easily. Further, the colors make the earplug structure 2 of the present invention more beautiful.

What is claimed is:

1. An earplug structure, being a columnar body, and having a sound insulation portion disposed at an end of the earplug structure, and a holding portion disposed at an another end of the earplug structure, characterized in that the sound insulation portion includes a first flap, a second flap, a third flap and a fourth flap which are bowl-shaped structures, and the first flap has a surface curvature from −6.275 to 5.167, and the second flap has a surface curvature from −6.211 to −4.233, and the third flap has a surface curvature from −4.53 to −4.335, and the fourth flap has a surface curvature from −3.963 to −3.872, and the first flap, the second flap, the third flap and the fourth flap come with different sizes, and the sizes increase gradually from the first flap to the fourth flap, and the earplug structure has a connecting hole formed at the end having the holding portion.

2. The earplug structure as claimed in claim 1, wherein the holding portion is doped with a metal powder therein.

3. The earplug structure as claimed in claim 2, wherein a gap between the first flap and the second flap, the second flap and the third flap, and the third flap and the fourth flap is 4 mm-4.5 mm.

4. The earplug structure as claimed in claim 3, wherein a volume ratio of the first flap to the second flap, the second flap to the third flap, and the third flap to the fourth flap is ranging from 1:1.25 to 1:1.43.

5. The earplug structure as claimed in claim 4, further comprising a sling, such that when a pair of earplug structures is used, both ends of the sling are inserted and fixed into the connecting hole of each of the earplug structures.

6. The earplug structure as claimed in claim 5, wherein the first flap, the second flap, the third flap and the fourth flap are made of a thermoplastic polyester plastic material.
